(19) United States
(12) Patent Application Publication
(10) Pub. No.: US 2007/0293879 A1
(43) Pub. Date: Dec. 20, 2007

(54) Tissue fixation devices including reaction causing agents

(75) Inventors: Steve G. Baker, Redmond, WA (US); Stefan J. M. Kraemer, Seattle, WA (US)

Correspondence Address: GRAYBEAL JACKSON HALEY LLP, Suite. 350, 155-108th Avenue N.E., Bellevue, WA, 98004-5973, US

(73) Assignee: EndoGastric Solutions, Inc.

(21) Appl. No.: 11/454,621

(22) Filed: Jun. 15, 2006

(51) Int. Cl. A61B 17/08

(52) U.S. Cl. 606/151

(57) ABSTRACT

A fastener for use in fastening tissue layers in a mammalian body comprises a first member, a second member, and a connecting member fixed to each of the first and second members intermediate their ends and extending there between. One of the first and second members has a through channel along its axis arranged to be slidingly received on a tissue piercing deployment wire, and a slit extending between the first and second ends and communicating with the through channel. The connecting member includes an agent comprising a plurality of elongated fibers emanating from and carried by the connecting member that stimulates reaction of the tissue layers.

### FIELD OF THE INVENTION

The present invention generally relates to tissue fixation devices. The present invention more particularly relates to tissue fixation devices which include agents that stimulate reactions, such as tissue reaction, for example, tissue growth, tissue healing, tissue scarring, and/or tissue adhesion, or anti-bacteria reactions.

### BACKGROUND

Tissue fixation devices or fasteners find many different uses. One use for such a device is in the treatment of GastroEsophageal Reflux Disease (GERD).

GERD is a chronic condition caused by the failure of the anti-reflux barrier located at the gastroesophageal junction to keep the contents of the stomach from splashing into the esophagus. The splashing is known as gastroesophageal reflux. The stomach acid is designed to digest meat, and will digest esophageal tissue when persistently splashed into the esophagus.

A principal reason for regurgitation associated with GERD is the mechanical failure of a deteriorated gastroesophageal flap to close and seal against high pressure in the stomach. Due to reasons including lifestyle, a Grade I normal gastroesophageal flap may deteriorate into a malfunctioning Grade III or absent valve Grade IV. With a deteriorated gastroesophageal flap, the stomach contents are more likely to be regurgitated into the esophagus, the mouth, and even the lungs. The regurgitation is referred to as “heartburn” because the most common symptom is a burning discomfort in the chest under the breastbone. Burning discomfort in the chest and regurgitation (burping up) of sour-tasting and/or bitter-tasting gastric juice into the mouth are classic symptoms of gastroesophageal reflux disease (GERD). When stomach acid is regurgitated into the esophagus, it is usually cleared quickly by esophageal contractions. Heartburn (backwashing of stomach acid and bile onto the esophagus) results when stomach acid is frequently regurgitated into the esophagus and the esophageal wall is inflamed.

Complications develop for some people who have GERD. Esophagitis (inflammation of the esophagus) with erosions and ulcerations (breaks in the lining of the esophagus) can occur from repeated and prolonged acid exposure. If these breaks are deep, bleeding or scarring of the esophagus with formation of a stricture (narrowing of the esophagus) can occur. If the esophagus narrows significantly, then food sticks in the esophagus and the symptom is known as dysphagia. GERD has been shown to be one of the most important risk factors for the development of esophageal adenocarcinoma. In a subset of people who have severe GERD, if acid exposure continues, the injured squamous lining is replaced by a precancerous lining (called Barrett's Esophagus) in which a cancerous esophageal adenocarcinoma can develop.

Other complications of GERD may not appear to be related to esophageal disease at all. Some people with GERD may develop recurrent pneumonia (lung infection), asthma (wheezing), or a chronic cough from acid backing up into the esophagus and all the way up through the upper esophageal sphincter into the lungs. In many instances, this occurs at night, while the person is in a supine position and sleeping. Occasionally, a person with severe GERD will be awakened from sleep with a choking sensation. Hoarseness can also occur due to acid reaching the vocal cords, causing a chronic inflammation or injury.

GERD never improves without intervention. Life style changes combined with both medical and surgical treatments exist for GERD. Medical therapies include antacids and proton pump inhibitors. However, the medical therapies only mask the reflux. Patients still get reflux and perhaps emphysema because of particles refluxed into the lungs. Barrett's esophagus results in about 10% of the GERD cases. The esophageal epithelium changes into tissue that tends to become cancerous from repeated acid washing despite the medication.

Several open laparotomy and laparoscopic surgical procedures are available for treating GERD. One surgical approach is the Nissen fundoplication. The Nissen approach typically involves a 360-degree wrap of the fundus around the gastroesophageal junction. The procedure has a high incidence of postoperative complications. The Nissen approach creates a 360-degree moveable flap without a fixed portion. Hence, Nissen does not restore the normal movable flap. The patient cannot burp because the fundus was used to make the repair, and may frequently experience dysphagia. Another surgical approach to treating GERD is the Belsey Mark IV (Belsey) fundoplication. The Belsey procedure involves creating a valve by suturing a portion of the stomach to an anterior surface of the esophagus. It reduces some of the postoperative complications encountered with the Nissen fundoplication, but still does not restore the normal movable flap. None of these procedures fully restores the normal anatomical anatomy or produces a normally functioning gastroesophageal junction.

Another surgical approach is the Hill repair. In the Hill repair, the gastroesophageal junction is anchored to the posterior abdominal areas, and a 180-degree valve is created by a system of sutures. The Hill procedure restores the moveable flap, the cardiac notch and the Angle of His. However, all of these surgical procedures are very invasive, regardless of whether done as a laparoscopic or an open procedure.

New, less surgically invasive approaches to treating GERD involve transoral endoscopic procedures. One procedure contemplates a machine device with robotic arms that is inserted transorally into the stomach. While observing through an endoscope, an endoscopist guides the machine within the stomach to engage a portion of the fundus with a corkscrew-like device on one arm. The arm then pulls on the engaged portion to create a fold of tissue or radial plication at the gastroesophageal junction. Another arm of the machine pinches the excess tissue together and fastens the excess tissue with one pre-tied implant. This procedure does not restore normal anatomy. The fold created does not have anything in common with a valve. In fact, the direction of the radial fold prevents the fold or plication from acting as a flap of a valve.

Another transoral procedure contemplates making a fold of fundus tissue near the deteriorated gastroesophageal flap to recreate the lower esophageal sphincter (LES). The procedure requires placing multiple U-shaped tissue clips around the folded fundus to hold it in shape and in place.

This and the previously discussed procedure are both highly dependent on the skill, experience, aggressiveness, and courage of the endoscopist. In addition, these and other procedures may involve esophageal tissue in the repair. Esophageal tissue is fragile and weak, in part due to the fact, that the esophagus is not covered by serosa, a layer of very sturdy, yet very thin tissue, covering and stabilizing all intraabdominal organs, similar like a fascia covering and stabilizing muscle. Involvement of esophageal tissue in the repair of a gastroesophageal flap valve poses unnecessary risks to the patient, such as an increased risk of fistulas between the esophagus and the stomach.

A new and improved apparatus and method for restoration of a gastroesophageal flap valve is fully disclosed in U.S. Pat. No. 6,790,214 which issued on Sep. 14, 2004, on an application filed May 17, 2002, for TRANSORAL ENDOSCOPIC GASTROESOPHAGEAL FLAP VALVE RESTORATION DEVICE, ASSEMBLY, SYSTEM AND METHOD, which is assigned to the assignee of this invention, and which is incorporated herein by reference. That apparatus and method provides a transoral endoscopic gastroesophageal flap valve restoration. A longitudinal member arranged for transoral placement into a stomach carries a tissue shaper that non-invasively grips and shapes stomach tissue. A tissue fixation device is then deployed to maintain the shaped stomach tissue in a shape approximating a gastroesophageal flap.

Whenever tissue is to be maintained in a shape as, for example, in the improved assembly last mentioned above, it is necessary to fasten at least two layers of tissue together. Once the tissue layers are fastened together, it is intended that the tissue layers adhere together. Some tissue combinations rarely exhibit adherence. Other tissue or tissue combinations eventually do adhere together but at varying rates. Generally, it is desired for the adherence to occur as quickly as possible. Further, some tissue and tissue combinations will be punctured or otherwise injured during the fastener deployment process. In these cases, it is desirable that the fastened tissue also heal and seal punctures that may be formed. Sometimes, the healing reaction process promotes tissue adhesion. Other tissue reactions that promote tissue adhesion include scarring and growth.

The present invention, in its broader aspects, addresses the issues of tissue or bacterial reactions following tissue fastening. Accordingly, as will be seen hereinafter, the various embodiments are directed to tissue fasteners including agents to promote tissue reactions such as, for example, growth, healing, sealing, scarring, fusion, and adhesion.

### SUMMARY

The invention provides a fastener arranged to secure at least two tissue layers together. The fastener comprises a first anchor member, a second anchor member, and a penetrating member connected to the first and second anchor members that penetrates the at least two tissue layers such that the first and second anchor members engage outermost surfaces of the at least two tissue layers with the penetrating member extending through the at least two tissue layers from the first anchor member to the second anchor member. The penetrating member includes an agent that stimulates a reaction at the at least two tissue layers.

The agent is arranged to stimulate a reaction of the tissue layers. The reactions may include at least one of growth, healing, sealing, scarring, fusion, and adhesion. The agent may include an antibiotic.

The agent may comprise a plurality of elongated fibers emanating from and carried by the penetrating member. The elongated fibers may be formed of one of silk, polyester, and polypropylene, and other natural and synthetic materials promoting controlled tissue reactions. The plurality of fibers may also be configured in loops. The elongated fibers may be arranged on the penetrating member such that at least some of the elongated fibers are between the at least two tissue layers when the fastener is deployed. The elongated fibers may further be biodegradable.

The agent may comprise a plurality of elongated fibers emanating from and carried by the penetrating member. The fibers may include a substance that elutes from the elongated fibers to stimulate reaction of the at least two tissue layers. The substance may be one of a powder and a coating.

The agent may comprise a plurality of elongated fibers emanating from and carried by the penetrating member, the elongated fibers carrying a substance that stimulates the reaction. The substance may be one of a powder and a coating.

The first and second anchor members may have first and second ends. The penetrating member maybe fixed to each of the first and second anchor members intermediate the first and second ends and extend between the first and second members. One of the first and second anchor members may have a longitudinal axis and a through channel along the axis arranged to be slidingly received on a tissue piercing deployment wire. The one of the first and second anchor members may have a slit extending between the first and second ends and communicating with the through channel.

The agent may be arranged to stimulate a reaction of the tissue layers including at least one of growth, healing, scarring, and adhesion. The agent may comprise a plurality of elongated fibers emanating from and carried by the penetrating member. The plurality of fibers may be configured in loops. The elongated fibers may be formed of one of silk, polyester, and polypropylene, and other natural and synthetic materials promoting controlled tissue reactions. The elongated fibers may be arranged on the penetrating member such that at least some of the elongated fibers are between the at least two tissue layers. The elongated fibers may be biodegradable. The agent may comprise a plurality of elongated fibers emanating from and carried by the penetrating member, the fibers including a substance that elutes from the elongated fibers to stimulate reaction of the at least two tissue layers. The eluting substance may be one of a powder and a coating. The agent may comprise a plurality of elongated fibers emanating from and carried by the penetrating member, the elongated fibers carrying a substance that stimulates the reaction. The substance may be one of a powder and a coating. The agent may be one of a powder and a coating.

In another embodiment, a fastener for use in fastening at least two tissue layers in a mammalian body comprises a first member, a second member, wherein the first and second members have first and second ends, and a connecting member fixed to each of the first and second members intermediate the first and second ends and extending between the first and second members. The first and second members are separated by the connecting member. One of the first and second members has a through channel along the axis arranged to be slidingly received on a tissue piercing deployment wire and a slit extending between the first and second ends and communicating with the through channel. The connecting member includes an agent that stimulates a reaction at the at least two tissue layers.

In a further embodiment, a fastener for use in fastening tissue layers in a mammalian body comprises a first member and a second member. The first and second members have first and second ends. A connecting member is fixed to each of the first and second members intermediate the first and second ends and extends between the first and second members. The first and second members are separated by the connecting member. One of the first and second members has a through channel along the axis arranged to be slidingly received on a tissue piercing deployment wire, and a slit extending between the first and second ends that communicates with the through channel. The connecting member includes an agent comprising a plurality of elongated fibers emanating from and carried by the connecting member that stimulates reaction of the tissue layers. The fastener and agent may both be absorbable/bio-degradable.

### BRIEF DESCRIPTION OF THE DRAWINGS

The features of the present invention which are believed to be novel are set forth with particularity in the appended claims. The invention, together with further objects and advantages thereof, may best be understood by making reference to the following description taken in conjunction with the accompanying drawings, in the several figures of which like reference numerals identify like elements, and wherein:

FIG. 1 is a front cross-sectional view of the esophago-gastro-intestinal tract from a lower portion of the esophagus to the duodenum;

FIG. 2 is a front cross-sectional view of the esophago-gastro-intestinal tract illustrating a Grade I normal appearance movable flap of the gastroesophageal valve (in dashed lines) and a Grade III reflux appearance gastroesophageal flap of the gastroesophageal valve (in solid lines);

FIG. 3 is an end plan view of a fastener embodying the present invention;

FIG. 4 is a side plan view of the fastener of FIG. 3;

FIG. 5 is a side view, with portions cut away, of the fastener FIG. 3 being delivered for deployment;

FIG. 6 is a side view, with portions cut away of the fastener of FIG. 3 after having been deployed;

FIG. 7 is an end plan view of another embodiment of the present invention;

FIG. 8 is a side plan view of the fastener of FIG. 7;

FIG. 9 is a side view, with portions cut away of the fastener of FIG. 7 being delivered for deployment;

FIG. 10 is a side view, with portions cut away, of the fastener of FIG. 7 after having been deployed;

FIG. 11 is an end plan view of another embodiment of the present invention;

FIG. 12 is a side plan view of the fastener of FIG. 11;

FIG. 13 is a side view, with portions cut away of the fastener of FIG. 11 being delivered for deployment; and

FIG. 14 is a side view, with portions cut away, of the fastener of FIG. 11 after having been deployed; and

FIG. 15 is a perspective view, with portions cut away, of a restored GEF being secured by fasteners embodying the invention.

### DETAILED DESCRIPTION

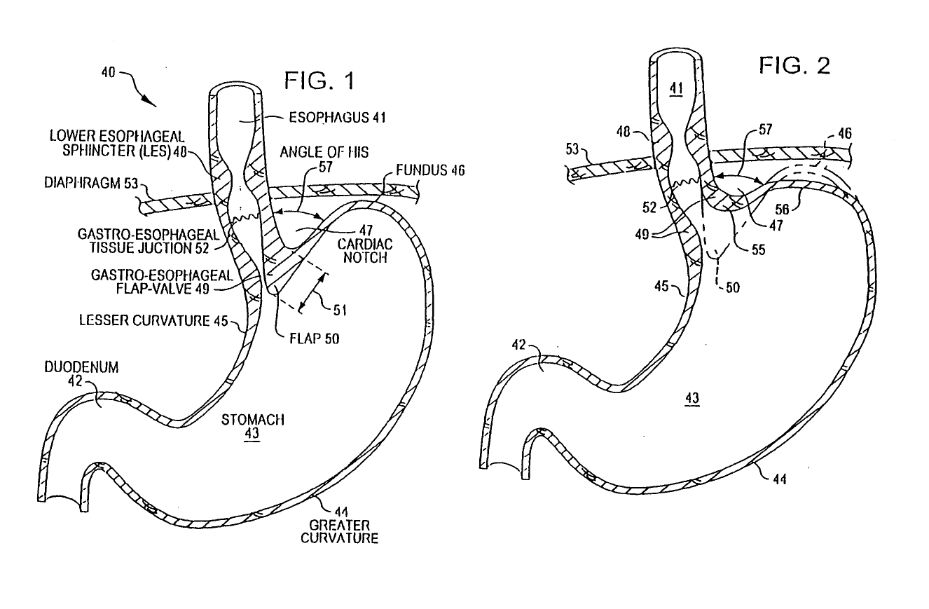

FIG. 1 is a front cross-sectional view of the esophageal-gastro-intestinal tract 40 from a lower portion of the esophagus 41 to the duodenum 42. The stomach 43 is characterized by the greater curvature 44 on the anatomical left side and the lesser curvature 45 on the anatomical right side. The tissue of the outer surfaces of those curvatures is referred to in the art as serosa tissue. As will be seen subsequently, the nature of the serosa tissue is used to advantage for its ability to bond to like serosa tissue. The fundus 46 of the greater curvature 44 forms the superior portion of the stomach 43, and traps gas and air bubbles. The esophageal tract 41 enters the stomach 43 at an esophageal orifice below the superior portion of the fundus 46, forming a cardiac notch 47 and an acute angle with respect to the fundus 46 known as the Angle of His 57. The lower esophageal sphincter (LES) 48 is a discriminating sphincter able to distinguish between burping gas, liquids, and solids, and works in conjunction with the fundus 46 to burp. The gastroesophageal valve (GEV) 49 includes a moveable portion and an opposing more stationary portion. The moveable portion of the GEV 49 is an approximately 180 degree, semicircular, gastroesophageal flap 50 (alternatively referred to as a “normal moveable flap” or “moveable flap”) formed of tissue at the intersection between the esophagus 41 and the stomach 43. The opposing more stationary portion of the GEV 49 comprises a portion of the lesser curvature 45 of the stomach 43 adjacent to its junction with the esophagus 41. The gastroesophageal flap 50 of the GEV 49 principally comprises tissue adjacent to the fundus 46 portion of the stomach 43, is about 4 to 5 cm long (51) at it longest portion, and the length may taper at its anterior and posterior ends. The gastroesophageal flap 50 is partially held against the lesser curvature 45 portion of the stomach 43 by the pressure differential between the stomach 43 and the thorax, and partially by the resiliency and the anatomical structure of the GEV 49, thus providing the valving function. The GEV 49 is similar to a flutter valve, with the gastroesophageal flap 50 being flexible and closeable against the other more stationary side.

The esophageal tract is controlled by an upper esophageal sphincter (UES) in the neck near the mouth for swallowing, and by the LES 48 and the GEV 49 at the stomach. The normal anti-reflux barrier is primarily formed by the LES 48 and the GEV 49 acting in concert to allow food and liquid to enter the stomach, and to considerably resist reflux of stomach contents into the esophagus 41 past the gastroesophageal tissue junction 52. Tissue aboral of the gastroesophageal tissue junction 52 is generally considered part of the stomach because the tissue protected from stomach acid by its own protective mechanisms. Tissue oral of the gastroesophageal junction 52 is generally considered part of the esophagus and it is not protected from injury by prolonged exposure to stomach acid. At the gastroesophageal junction 52, the juncture of the stomach and esophageal tissues form a zigzag line, which is sometimes referred to as the “Z-line.” For the purposes of these specifications, including the claims, “stomach” means the tissue aboral of the gastroesophageal junction 52.

FIG. 2 is a front cross-sectional view of an esophago-gastro-intestinal tract illustrating a Grade I normal appearance movable flap 50 of the GEV 49 (shown in dashed lines) and a deteriorated Grade III gastroesophageal flap 55 of the GEV 49 (shown in solid lines). As previously mentioned, a principal reason for regurgitation associated with GERD is the mechanical failure of the deteriorated (or reflux appearance) gastroesophageal flap 55 of the GEV 49 to close and seal against the higher pressure in the stomach. Due to reasons including lifestyle, a Grade I normal gastroesophageal flap 50 of the GEV 49 may deteriorate into a Grade III deteriorated gastroesophageal flap 55. The anatomical results of the deterioration include moving a portion of the esophagus 41 that includes the gastroesophageal junction 52 and LES 48 toward the mouth, straightening of the cardiac notch 47, and increasing the Angle of His 57. This effectively reshapes the anatomy aboral of the gastroesophageal junction 52 and forms a flattened fundus 56. The deteriorated gastroesophageal flap 55 illustrates a gastroesophageal flap valve 49 and cardiac notch 47 that have both significantly degraded. Dr. Hill and colleagues developed a grading system to describe the appearance of the GEV and the likelihood that a patient will experience chronic acid reflux. L. D. Hill, et al., The gastroesophageal flap valve: in vitro and in vivo observations, Gastrointestinal Endoscopy 1996:44:541-547. Under Dr. Hill's grading system, the normal movable flap 50 of the GEV 49 illustrates a Grade I flap valve that is the least likely to experience reflux. The deteriorated gastroesophageal flap 55 of the GEV 49 illustrates a Grade III (almost Grade IV) flap valve. A Grade IV flap valve is the most likely to experience reflux. Grades II and III reflect intermediate grades of deterioration and, as in the case of III, a high likelihood of experiencing reflux. With the deteriorated GEV represented by deteriorated gastroesophageal flap 55 and the fundus 46 moved inferior, the stomach contents are presented a funnel-like opening directing the contents into the esophagus 41 and the greatest likelihood of experiencing reflux. Disclosed subsequently are fasteners and assemblies which may be employed to advantage in restoring the normal gastroesophageal valve anatomy.

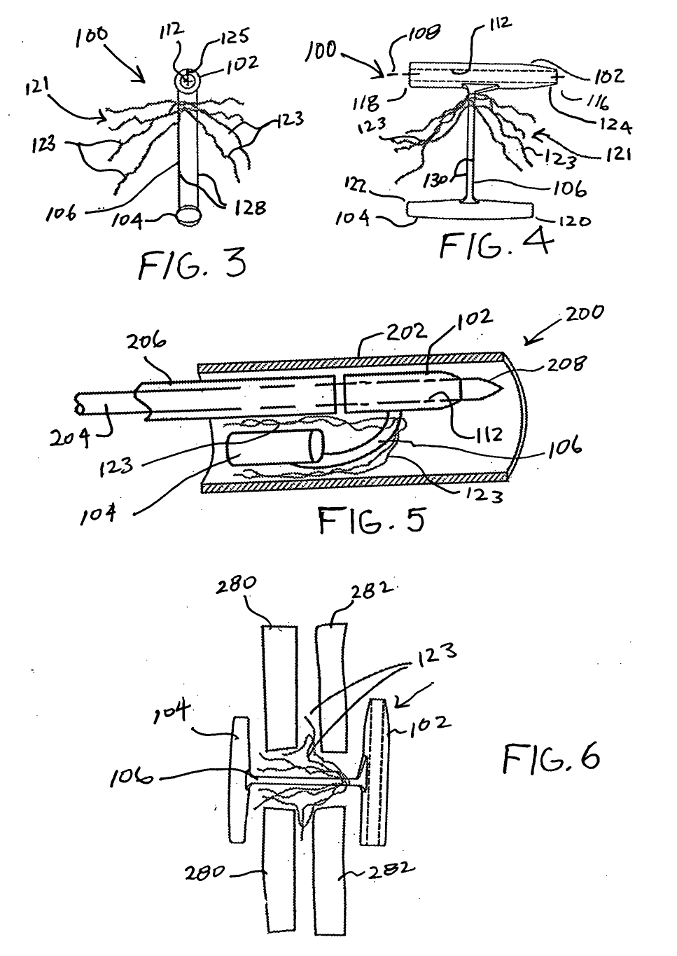

FIG. 3 is an end plan view and FIG. 4 is a side plan view of a fastener 100 embodying the present invention. The fastener 100 generally includes a first anchor member 102, a second anchor member 104, and a penetrating member 106. As may be noted in FIG. 3, the first anchor member 102 and second anchor member 104 are substantially parallel to each other and substantially perpendicular to the penetrating member 106 which connects the first anchor member 102 to the second anchor member 104. Parts or all of the fasteners may be absorbable.

The first anchor member 102 is generally cylindrical or can have any other shape. It has a longitudinal axis 108 and a through channel 112 along the longitudinal axis 108. The through channel 112 is formed by a through bore which is dimensioned to be slidingly received on a tissue piercing deployment wire to be described.

The first anchor member 102 also includes a first end 116 and a second end 118. Similarly, the second anchor member 104 includes a first end 120 and a second end 122. The first end 116 of member 102 forms a pointed dilation tip 124. The dilation tip 124 may be conical and more particularly takes the shape of a truncated cone. The tip can also be shaped to have a cutting edge in order to reduce tissue resistance. It may also have a “ski-tip” or other directing configurations.

The first and second anchor members 102 and 104 and the penetrating member 106 may be formed of different materials and have different textures. These materials may include, for example, plastic materials such as polypropylene, polyethylene, polyglycolic acid, polyurethane, or a thermoplastic elastomer. The plastic materials may include a pigment contrasting with body tissue color to enable better visualization of the fastener during its deployment. Alternatively, the fastener may be formed of a metal, such as stainless steel or a shape memory metal, such as Nitinol or a shape memory plastic. The fasteners may also contain radio opaque substances and parts.

As may be further noted in FIGS. 3 and 4, the penetrating member 106 has a vertical dimension 128 and a horizontal dimension 130 which is transverse to the vertical dimension. The horizontal dimension is substantially less than the vertical dimension to render the penetrating member 106 readily bendable in a horizontal plane. The penetrating member is further rendered bendable by the nature of the material from which the fastener 100 is formed. The penetrating member may be formed from either an elastic plastic or a permanently deformable plastic. An elastic material would prevent compression necrosis in some applications.

It may be noted in FIGS. 3 and 4, that the first anchor member 102 has a continuous lengthwise slit 125 extending between the first and second ends 116 and 118. The slit 125 communications with the through channel 112. Also, because the first anchor member 102 is formed of flexible material, the slit 125 may be made larger through separation to allow the deployment wire to be snapped into and released from the through channel 112. This permits ready release of the first anchor member 102 during deployment and decreases compression on the tissue layers. For a complete description of a manner in which the fastener 100 may be deployed to obtain quick release of the fastener from the tissue piercing deployment wire, reference may be had to co-pending U.S. application Ser. No. 11/043,903 filed on Jan. 25, 2005, titled SLITTED TISSUE FIXATION DEVICES AND ASSEMBLIES FOR DEPLOYING THE SAME, and which is incorporated herein by reference. The slit 125 extends substantially parallel to the through channel 112 and the center axis 108 of the first anchor member 102. The slit 125 has a width dimension that is smaller or less than the diameter of the through channel 112. This assures that the fastener 100 will remain on a tissue piercing deployment wire as it is pushed towards and into the tissue during deployment.

With continued reference to FIGS. 3 and 4, and in accordance with this embodiment of the present invention, the device 100 further includes an agent 121 that promotes tissue reaction after the fastener is deployed. The tissue reaction may include any one or combination of growth, scarring, healing, sealing, fusion, and adhesion.

The agent 121, in accordance with this embodiment, comprises a plurality of elongated fibers 123. The fibers may be formed of silk, polyester, polypropylene, carbon, nylon, and/or a biodegradable material such as polyglycolic acid, polyglactin or polydioxanone. Alternatively, the fibers may include, by being formed from or carrying, a substance that elutes into the tissue after deployment to stimulate tissue reaction(s). Such substances may be calcium alginate, sodium alginate, corn starch, talc, latex, and a human growth hormone, such as somatropin, for example. To carry the substances that promote tissue reaction, the substances may form a coating on the fibers 123 either in a continuous form or a powder form. Substances which may form a continuous coating include calcium alginate gel, sodium alginate, and collagen for example. Substances which may be carried by the fibers in powered form include corn starch, glove powder, and silver, for example. Other materials that may be employed include healing agents such as antibiotics, as for example, vancomycin, or tissue adhesives, such as fibrin.

The fibers may be attached to the fastener 100 by being attached, for example, to the penetrating member 106, in accordance with this embodiment. The fibers may be attached to the penetrating member 106 by tying, by trapping them in a slit made in the penetrating member, by melting a portion of the penetrating member about the fibers, or by adhesion with an adhesive. Other forms of attachment are possible.

Either one or both of the fastener and the fibers may be biodegradable. In some cases, it may be preferable to have the fastener be biodegradable and the fibers prevalent.

Alternatively, the agent may not require the fibers 123 and instead simply be a substance formed in the fastener or a substance carried on the fastener 100 without the fibers 121. However, inclusion of the fibers is advantageous because they present an increased surface area to the fastened tissue to accelerate the tissue reaction process. Other forms of surface area enhancement are also possible. The use of fibers, however, is particularly advantageous because they can be readily tucked away so as to not interfere with the fastener deployment process as will be seen in FIG. 5.

Referring now to FIG. 5, it is a perspective view with portions cut away of a fastener assembly 200 for deploying the fastener 100. The assembly 200 generally includes a stylet or tissue piercing deployment wire 204, a pusher 206, and a guide channel or tube 202.

The first anchor member 102 of the fastener 100 is slidingly received on the deployment wire 204. The deployment wire 204 has a pointed tip 208 for piercing the tissue layers 280 and 282 (FIG. 6) to be fastened together. As will be further noted in FIG. 5, the second anchor member 104 is disposed along side the first anchor member 102. This is rendered possible by the flexibility and configuration of the penetrating member 106. The fibers 123 are tucked in the guide channel and extend along side the penetrating member 106. The fastener 100 is now ready for deployment. It may be deployed as fully described in the aforementioned co-pending application Ser. No. 11/043,903.

When the fastener is fully deployed, the first anchor member 102 and second anchor member 104 engage the outermost or innermost surfaces 283 and 281 respectively of the tissue layers 282 and 280 respectively with the penetrating member 106 extending through the tissue there between. This may be seen in FIG. 6. Here it may be seen that the first anchor member 102 is adjacent outer surface 283 of the tissue layer 282, the second anchor member 104 is adjacent outer surface 281 of the tissue layer 281, and that the penetrating member 206 extends between the tissue layers. The fibers 123 fill in the tissue penetration path or between the tissue layers in broad surface area contact therewith to stimulate reaction of the fastened tissue as previously described.

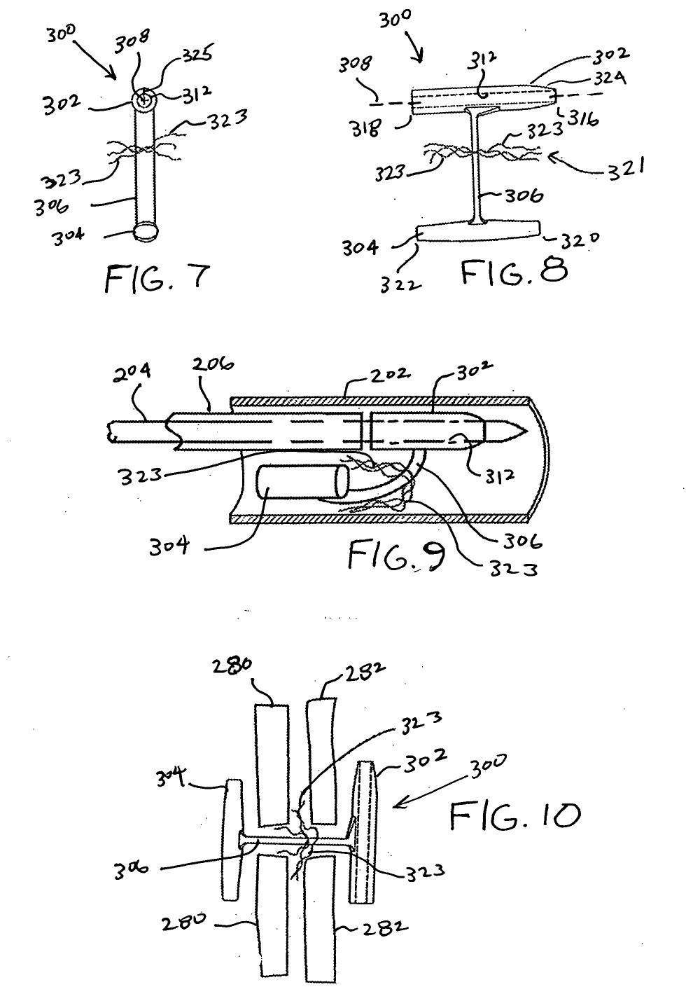

FIG. 7 is an end plan view and FIG. 8 is a side plan view of another fastener 300 embodying the present invention. The fastener 300, as in the previous embodiment, generally includes a first anchor member 302, a second anchor member 304, and a penetrating member 306. As may be noted in FIG. 7, the first anchor member 302 and second anchor member 304 are substantially parallel to each other and substantially perpendicular to the penetrating member 306 which connects the first anchor member 302 to the second anchor member 304.

The first anchor member 302 is again generally cylindrical. It has a longitudinal axis 308 and a through channel 312 along the longitudinal axis 308. The through channel 312 is formed by a through bore which is dimensioned to be slidingly received on the tissue piercing deployment wire.

The first anchor member 302 also includes a first end 316 and a second end 318. Similarly, the second anchor member 304 includes a first end 320 and a second end 322. The first end 316 of member 302 forms a pointed dilation tip 324. The dilation tip 324 again may be conical or shaped otherwise and more particularly take the shape of a truncated cone.

The first and second anchor members 302 and 304 and the penetrating member 306 may be formed of any of the different materials and have the different textures previously described. As in the embodiment of FIGS. 3 and 4, the penetrating member 306 has a horizontal dimension that is substantially less than its vertical dimension to render the penetrating member 306 readily bendable. The penetrating member is further rendered bendable by the nature of the material from which the fastener 300 is formed.

The first anchor member 302 has a continuous lengthwise slit 325 extending between the first and second ends 316 and 318. The slit 325 communicates with the through channel 312. Also, as in the previous embodiment, the slit 125 may be made larger through separation to allow the deployment wire to be snapped into and released from the through channel 312. This permits ready release of the first anchor member 302 during deployment and decreases compression on the tissue layers. As in the previous embodiment, the fastener 300 may be deployed as described in copending U.S. application Ser. No. 11/043,903.

With continued reference to FIGS. 7 and 8, and in accordance with this embodiment of the present invention, the device 300 further includes an agent 321 that promotes tissue reaction or serves as tissue glue after the fastener is deployed. Again, the tissue reaction may include any one or combination of growth, scarring, healing, fusion, and adhesion.

The agent 121, in accordance with this embodiment, comprises a plurality of elongated fibers 323 positioned substantially at the midpoint between the first anchor member 302 and the second anchor member 304. This permits the fibers to fill in between the fastened tissue layers and/or the fastener path while allowing the fibers to be shorter in length. The fibers may be formed of any of the substances and have any of the characteristics previously described with respect to the embodiment of FIGS. 3 and 4. The fibers may also be attached to the penetrating member 306 by tying, by trapping them in a slit made in the penetrating member, by melting a portion of the penetrating member about the fibers, or by adhesion with an adhesive.

FIG. 9, is a perspective view with portions cut away of the fastener assembly 200 delivering the fastener 300 for deployment. The first anchor member 302 of the fastener 300 is slidingly received on the deployment wire 204. The fibers 323 also extend along side the penetrating member 306. The fastener may now be deployed in a manner as fully described in the aforementioned co-pending application Ser. No. 11/043,903.

FIG. 10 shows the fastener 300 fully deployed. The first anchor member 302 and second anchor member 304 engage the outer surfaces 283 and 281 of the tissue layers 282 and 280 respectively with the penetrating member 306 extending through the tissue there between. The fibers 323 fill in between the tissue layers in broad surface area contact therewith to stimulate reaction of the fastened tissue as previously described.

FIG. 11 is an end plan view and FIG. 12 is a side plan view of still another fastener 400 embodying the present invention. The fastener 400, as in the previous embodiments, generally includes a first anchor member 402, a second anchor member 404, and a penetrating member 406. As may be noted in FIG. 12, the first anchor member 402 and second anchor member 404 are substantially parallel to each other and substantially perpendicular to the penetrating member 406 which connects the first anchor member 402 to the second anchor member 404. The fastener may be formed of any of the materials previously described with respect to fasteners 100 and 300 and may be similarly configured.

In accordance with this embodiment of the present invention, the agent 421 to promote tissue reaction after the fastener is deployed takes the form of a plurality of fiber loops 423. The fiber loops not only promote tissue reaction such as any one or combination of growth, scarring, healing, fusion, and adhesion, but in addition, promote attachment of ingrown tissue.

The fiber loops 423 are positioned substantially at the midpoint between the first anchor member 402 and the second anchor member 404. This again positions the fibers for filling in between the fastened tissue layers. The fiber loops may be formed of any of the substances and have any of the characteristics previously described with respect to the previous embodiments. The fiber loops may also be attached to the penetrating member 406 by tying, by trapping them in a slit made in the penetrating member, by melting a portion of the penetrating member about the fibers, or by adhesion with an adhesive.

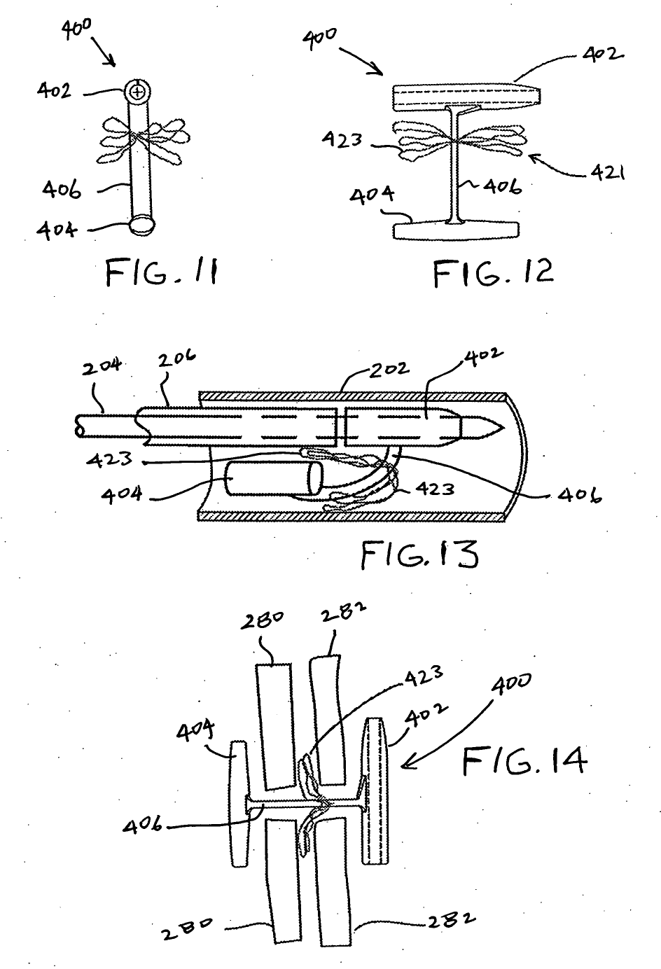

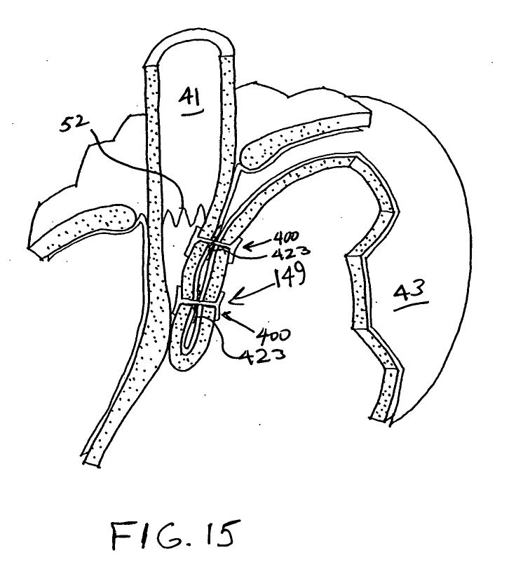

FIG. 13, is a perspective view with portions cut away of the fastener assembly 200 delivering the fastener 400 for deployment. The first anchor member 402 of the fastener 400 is slidingly received on the deployment wire 204. The fiber loops 423 also extend along side the penetrating member 406. The fastener may now be deployed in a manner as fully described in the aforementioned co-pending application Ser. No. 11/043,903.

FIG. 14 shows the fastener 400 fully deployed. The first anchor member 402 and second anchor member 404 engage the outer or inner surfaces 283 and 281 of the tissue layers 282 and 280 respectively with the penetrating member 406 extending through the tissue there between. The fiber loops 423 fill in between the tissue layers in broad surface area contact therewith to stimulate reaction of the fastened tissue and attachment of ingrowing tissue as previously described.

FIG. 15 shows a restored GEF 149. The GEF may be restored by practicing the method fully described in co-pending U.S. application Ser. No. 11/172,427, filed Jun. 29, 2005 for APPARATUS AND METHOD FOR MANIPULATING STOMACH TISSUE AND TREATING GASTROESOPHAGEAL REFLUX DISEASE, incorporated herein by reference. The GEF 149 may be maintained by a plurality of fasteners embodying the present invention. More specifically, as may be noted in FIG. 15, the restored GEF 149 is being maintained by fasteners 400 having fiber loops 423 to promote reaction of the fastened stomach tissue.

While the invention has been described by means of specific embodiments and applications thereof, it is understood that numerous modifications and variations may be made thereto by those skilled in the art without departing from the spirit and scope of the invention. It is therefore to be understood that within the scope of the claims, the invention may be practiced otherwise than as specifically described herein.

What is claimed:

1. A fastener arranged to secure at least two tissue layers together comprising:
a first anchor member;
a second anchor member; and
a penetrating member connected to the first and second anchor members that penetrates the at least two tissue layers such that the first and second anchor members engage outermost surfaces of the at least two tissue layers with the penetrating member extending through the at least two tissue layers from the first anchor member to the second anchor member, the penetrating member including an agent that stimulates a reaction at the at least two tissue layers.

2. The fastener of claim 1 wherein the agent includes an antibiotic.

3. The fastener of claim 1, wherein the agent is arranged to stimulate a reaction of the tissue layers including at least one of growth, healing, sealing, scarring, fusion, and adhesion.

4. The fastener of claim 1, wherein the agent comprises a plurality of elongated fibers emanating from and carried by the penetrating member.

5. The fastener of claim 4, wherein the elongated fibers are formed of one of silk, polyester, and polypropylene.

6. The fastener of claim 4, wherein the plurality of fibers are configured in loops.

7. The fastener of claim 4, wherein the elongated fibers are arranged on the penetrating member such that at least some of the elongated fibers are between the at least two tissue layers.

8. The fastener of claim 4, wherein at least one of the elongated fibers and fastener is biodegradable.

9. The fastener of claim 1, wherein the agent comprises a plurality of elongated fibers emanating from and carried by the penetrating member, the fibers including a substance that elutes from the elongated fibers to stimulate reaction of the at least two tissue layers.

10. The fastener of claim 9, wherein the substance is one of a powder and a coating.

11. The fastener of claim 1, wherein the agent comprises a plurality of elongated fibers emanating from and carried by the penetrating member, the elongated fibers carrying a substance that stimulates the reaction.

12. The fastener of claim 11, wherein the substance is one of a powder and a coating.

13. The fastener of claim 1, wherein the first and second anchor members have first and second ends, wherein the penetrating member is fixed to each of the first and second anchor members intermediate the first and second ends and extends between the first and second members, and wherein one of the first and second anchor members has a longitudinal axis and a through channel along the axis arranged to be slidingly received on a tissue piercing deployment wire.

14. The fastener of claim 13, wherein the one of the first and second anchor members has a slit extending between the first and second ends and communicating with the through channel.

15. The fastener of claim 14, wherein the agent is arranged to stimulate a reaction of the tissue layers including at least one of growth, healing, sealing, scarring, fusion, and adhesion.

16. The fastener of claim 14, wherein the agent comprises a plurality of elongated fibers emanating from and carried by the penetrating member.

17. The fastener of claim 16, wherein the plurality of fibers are configured in loops.

18. The fastener of claim 16, wherein the elongated fibers are formed of one of silk, polyester, and polypropylene.

19. The fastener of claim 16, wherein the elongated fibers are arranged on the penetrating member such that at least some of the elongated fibers are between the at least two tissue layers.

20. The fastener of claim 16, wherein the elongated fibers are biodegradable.

21. The fastener of claim 14, wherein the agent comprises a plurality of elongated fibers emanating from and carried by the penetrating member, the fibers including a substance that elutes from the elongated fibers to stimulate reaction of the at least two tissue layers.

22. The fastener of claim 21, wherein the substance is one of a powder and a coating.

23. The fastener of claim 14, wherein the agent comprises a plurality of elongated fibers emanating from and carried by the penetrating member, the elongated fibers carrying a substance that stimulates the reaction.

24. The fastener of claim 23, wherein the substance is one of a powder and a coating.

25. The fastener of claim 1, wherein the agent is one of a powder and a coating.

26. A fastener for use in fastening at least two tissue layers in a mammalian body, comprising:
a first member;
a second member,
the first and second members having first and second ends; and
a connecting member fixed to each of the first and second members intermediate the first and second ends and extending between the first and second members,
the first and second members being separated by the connecting member,
one of the first and second members having a through channel along the axis arranged to be slidingly received on a tissue piercing deployment wire, and a slit extending between the first and second ends and communicating with the through channel and
the connecting member including an agent that stimulates a reaction at the at least two tissue layers.

27. The fastener of claim 26 wherein the agent comprises an antibiotic.

28. The fastener of claim 26, wherein the agent is arranged to stimulate a reaction of the tissue layers including at least one of growth, healing, sealing, scarring, fusion, and adhesion.

29. The fastener of claim 26, wherein the agent comprises a plurality of elongated fibers emanating from and carried by the connecting member.

30. The fastener of claim 29, wherein the elongated fibers are formed of one of silk, polyester, and polypropylene.

31. The fastener of claim 29, wherein the plurality of fibers are configured in loops.

32. The fastener of claim 29, wherein the elongated fibers are arranged on the connecting member such that at least some of the elongated fibers are between the at least two tissue layers.

33. The fastener of claim 29, wherein at least one of the elongated fibers and fastener is biodegradable.

34. The fastener of claim 26, wherein the agent comprises a plurality of elongated fibers emanating from and carried by the connecting member, the fibers including a substance that elutes from the elongated fibers to stimulate reaction of the at least two tissue layers.

35. The fastener of claim 34, wherein the substance is one of a powder and a coating.

36. The fastener of claim 26, wherein the agent comprises a plurality of elongated fibers emanating from and carried by the connecting member, the elongated fibers carrying a substance that stimulates the reaction.

37. The fastener of claim 36, wherein the substance is one of a powder and a coating.

38. The fastener of claim 26 wherein the agent in one of a powder and a coating.

39. A fastener for use in fastening tissue layers in a mammalian body, comprising:
a first member;
a second member,
the first and second members having first and second ends; and
a connecting member fixed to each of the first and second members intermediate the first and second ends and extending between the first and second members,
the first and second members being separated by the connecting member,
one of the first and second members having a through channel along the axis arranged to be slidingly received on a tissue piercing deployment wire, and a slit extending between the first and second ends and communicating with the through channel and
the connecting member including an agent comprising a plurality of elongated fibers emanating from and carried by the connecting member that stimulates reaction of the tissue layers.
